(19) United States
(12) Patent Application Publication
(10) Pub. No.: US 2022/0220065 A1
(43) Pub. Date: Jul. 14, 2022

(54) ADDITIVE FOR ELECTROPLATING SOLUTIONS, ELECTROPLATING SOLUTION, ELECTROPLATING METHOD AND NOVEL COMPOUND

(71) Applicant: ADEKA CORPORATION, Tokyo (JP)

(72) Inventors: Takuya TAKAHASHI, Tokyo (JP); Shinya ISHIWATA, Tokyo (JP); Tomoko HATSUKADE, Tokyo (JP)

(73) Assignee: ADEKA CORPORATION, Tokyo (JP)

(21) Appl. No.: 17/614,098

(22) PCT Filed: May 18, 2020
(86) PCT No.: PCT/JP2020/019591

§ 371 Date: Nov. 24, 2021

(30) Foreign Application Priority Data
May 29, 2019 (JP) 2019-100090

(51) Int. Cl. C07C 237/10; C25D 3/38

(52) U.S. Cl. CPC C07C 237/10; C25D 3/38

(57) ABSTRACT

Provided is an additive for an electroplating solution, including a compound represented by the following general formula (1):

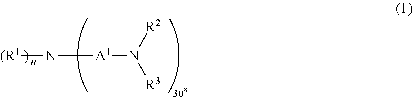

where R¹ to R³ each independently represent a group represented by the following general formula (2), A¹ represents an alkanediyl group having 2 to 4 carbon atoms, and “n” represents 0 or 1:

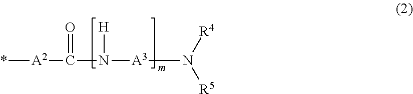

where R⁴ and R⁵ each independently represent a hydrogen atom or an alkyl group having 1 to 4 carbon atoms, A² and A³ each independently represent an alkanediyl group having 2 to 4 carbon atoms, “m” represents an integer of from 1 to 4, and * represents a bonding site.

### TECHNICAL FIELD

The present invention relates to an additive for an electroplating solution including a compound having a specific structure, an electroplating solution including the additive for an electroplating solution, an electroplating method including using the electroplating solution, and a novel compound.

### BACKGROUND ART

In the formation of a fine wiring, a through silicon via (TSV), and a bump in a highly integrated electronic circuit, an approach including filling a metal in a pattern, such as a groove or a hole, has heretofore been used. Electroplating is one typical approach including filling a metal. Of such approaches, copper electroplating including filling copper as a metal has been widely known. In the formation of a circuit by the copper electroplating, to obtain high connection reliability, satisfactory finishing is required in terms of, for example, the flatness of the surface of the circuit and the uniformity of the height thereof. A promoter, an inhibitor, a leveling agent, or the like is added to an electroplating solution for controlling, for example, the flatness of the surface and the uniformity of the height.

In recent years, in a process for the formation of the copper layer of an electronic device, an electroplating method including forming the copper layer at high speed and an electroplating solution suitable for the method have been required for a cost reduction and an improvement in productivity. In particular, when the shortening of the time period of the process is strictly required, there has arisen a need for increasing a current density to the vicinity of the limit current density at which the supply of a copper ion in the electroplating solution serves as a rate-determining step to preclude the deposition of copper. However, when an electroplating solution containing a conventional additive is used, there has been a large problem in that as the current density approaches the limit current density, the flatness of the upper surface of the copper layer is lost or a defect occurs in the side wall of the copper layer to adversely affect connection reliability.

In view of the foregoing, in Patent Document 1, there is a disclosure of the addition of a leveling agent, such as polyethyleneimine or polyvinylpyrrolidone, to a copper electroplating aqueous solution for filling a fine copper wiring.

### CITATION LIST

### Patent Document

Patent Document 1: JP 5809055 B2

### SUMMARY OF INVENTION

### Technical Problem

However, when electroplating is performed at high speed with the electroplating solution described in Patent Document 1 described above having added thereto the leveling agent, such as polyethyleneimine, there has been a problem in that a metal layer excellent in surface flatness cannot be obtained, and a defect occurs in the side wall of a metal layer. Accordingly, there has been required an additive for an electroplating solution capable of providing a metal layer, which is reduced in number of defects occurring in its side wall and is hence excellent in surface flatness. In particular, there has been required an additive for an electroplating solution capable of providing a metal layer, which is reduced in number of defects occurring in its side wall and is hence excellent in surface flatness, even when a current density is high.

In addition, in Patent Document 1 described above, there is no disclosure or suggestion of an effect in the case where an additive for an electroplating solution of the present invention and an electroplating solution including the additive are used.

### Solution to Problem

The inventors of the present invention have made investigations, and as a result, have found that the problems can be solved by using a compound having a specific structure as an additive for an electroplating solution. Thus, the inventors have reached the present invention.

That is, according to one embodiment of the present invention, there is provided an additive for an electroplating solution, including a compound represented by the following general formula (1):

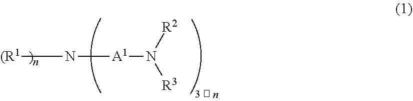

where R¹ to R³ each independently represent a group represented by the following general formula (2), A¹ represents an alkanediyl group having 2 to 4 carbon atoms, and “n” represents 0 or 1:

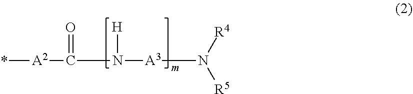

where R⁴ and R⁵ each independently represent a hydrogen atom or an alkyl group having 1 to 4 carbon atoms, A² and A³ each independently represent an alkanediyl group having 2 to 4 carbon atoms, “m” represents an integer of from 1 to 4, and * represents a bonding site.

In addition, according to one embodiment of the present invention, there is provided an electroplating solution, including the additive for an electroplating solution.

Further, according to one embodiment of the present invention, there is provided an electroplating method, including using the electroplating solution.

Further, according to one embodiment of the present invention, there is provided a compound represented by the following general formula (3):

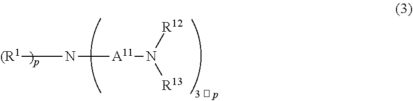

where R¹¹ to R¹³ each independently represent a group represented by the following general formula (4), An represents an alkanediyl group having 2 to 4 carbon atoms, and “p” represents 0 or 1:

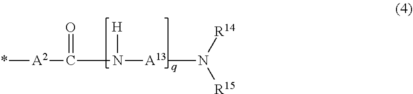

where R¹⁴ and R¹⁵ each independently represent a hydrogen atom or an alkyl group having 1 to 4 carbon atoms, A¹² and A¹³ each independently represent an alkanediyl group having 2 to 4 carbon atoms, “q” represents an integer of from 1 to 4, and * represents a bonding site, provided that when A¹¹ represents an alkanediyl group having 2 carbon atoms, “q” represents an integer of from 2 to 4.

### Advantageous Effects of Invention

According to the present invention, the additive for an electroplating solution capable of providing a metal layer, which is reduced in number of defects occurring in its side wall and is hence excellent in surface flatness, can be provided.

### BRIEF DESCRIPTION OF DRAWINGS

FIG. 1 is a schematic sectional view of a base to be plated after the formation of a copper layer on the surface of the base to be plated by an electroplating method in an evaluation test.

### DESCRIPTION OF EMBODIMENTS

<Additive for Electroplating Solution>

An additive for an electroplating solution of the present invention includes a compound represented by the general formula (1).

In the general formula (1), R¹ to R³ each independently represent a group represented by the general formula (2), A¹ represents an alkanediyl group having 2 to 4 carbon atoms, and “n” represents 0 or 1. Examples of the alkanediyl group having 2 to 4 carbon atoms that is represented by A¹ include an ethylene group, a propylene group, and a butylene group. A¹ preferably represents an ethylene group or a propylene group because a metal layer that is more excellent in surface flatness can be formed.

In the general formula (2), R⁴ and R⁵ each independently represent a hydrogen atom or an alkyl group having 1 to 4 carbon atoms, A² and A³ each independently represent an alkanediyl group having 2 to 4 carbon atoms, “m” represents an integer of from 1 to 4, and * represents a bonding site. Examples of the alkyl group having 1 to 4 carbon atoms that is represented by any one of R⁴ and R⁵ include a methyl group, an ethyl group, a n-propyl group, an isopropyl group, a n-butyl group, an isobutyl group, and a tertiary butyl group. Examples of the alkanediyl group having 2 to 4 carbon atoms that is represented by any one of A² and A³ include an ethylene group, a propylene group, and a butylene group. R⁴ and R⁵ each preferably represent a hydrogen atom or a methyl group, and A² and A³ each preferably represent an ethylene group because a metal layer that is more excellent in surface flatness can be formed.

Preferred specific examples of the compound represented by the general formula (1) include the following compounds No. 1 to No. 24. In the following compounds, the symbol “Me” represents a methyl group, and the symbol “Et” represents an ethyl group.

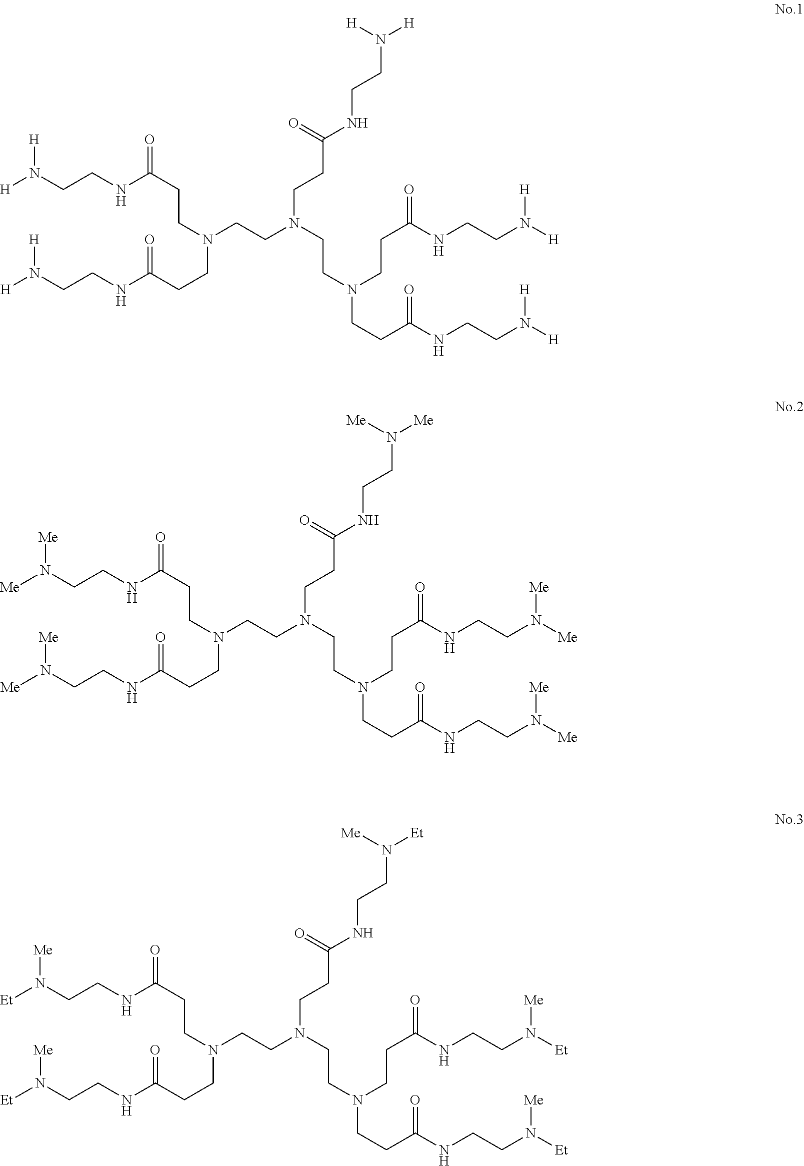

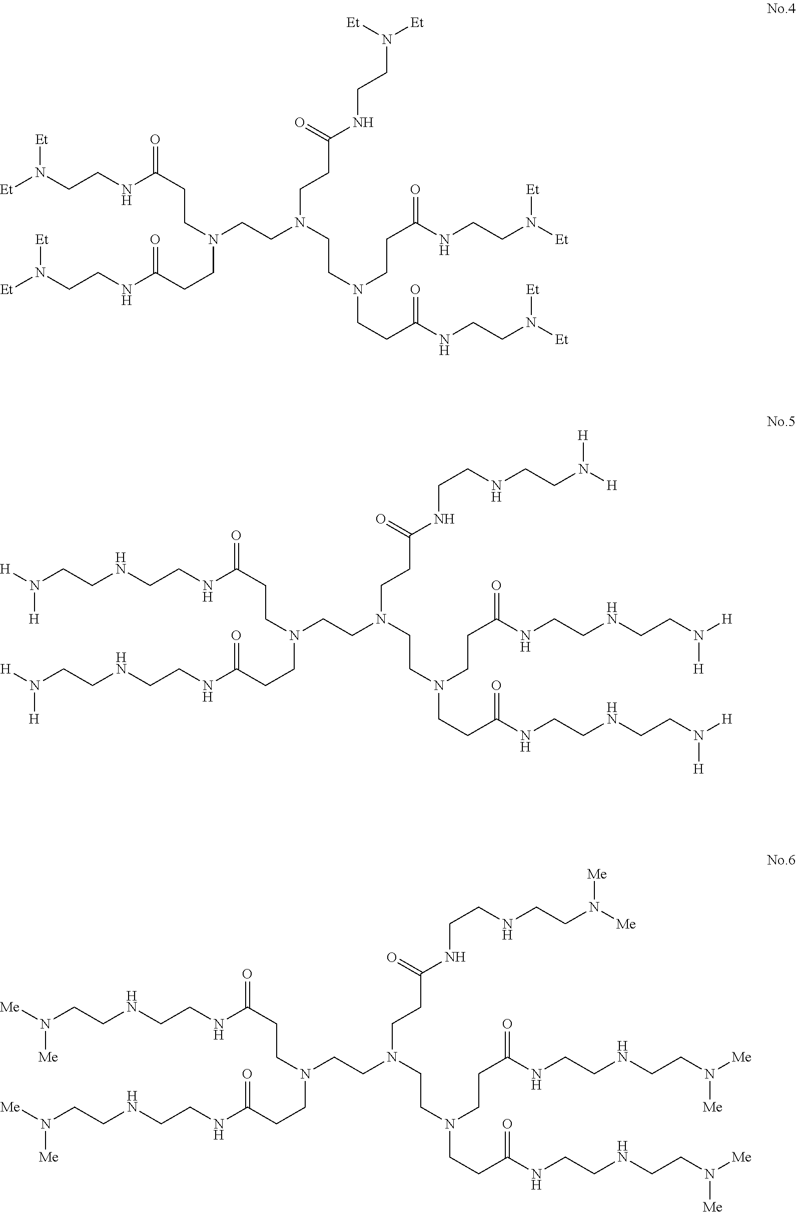

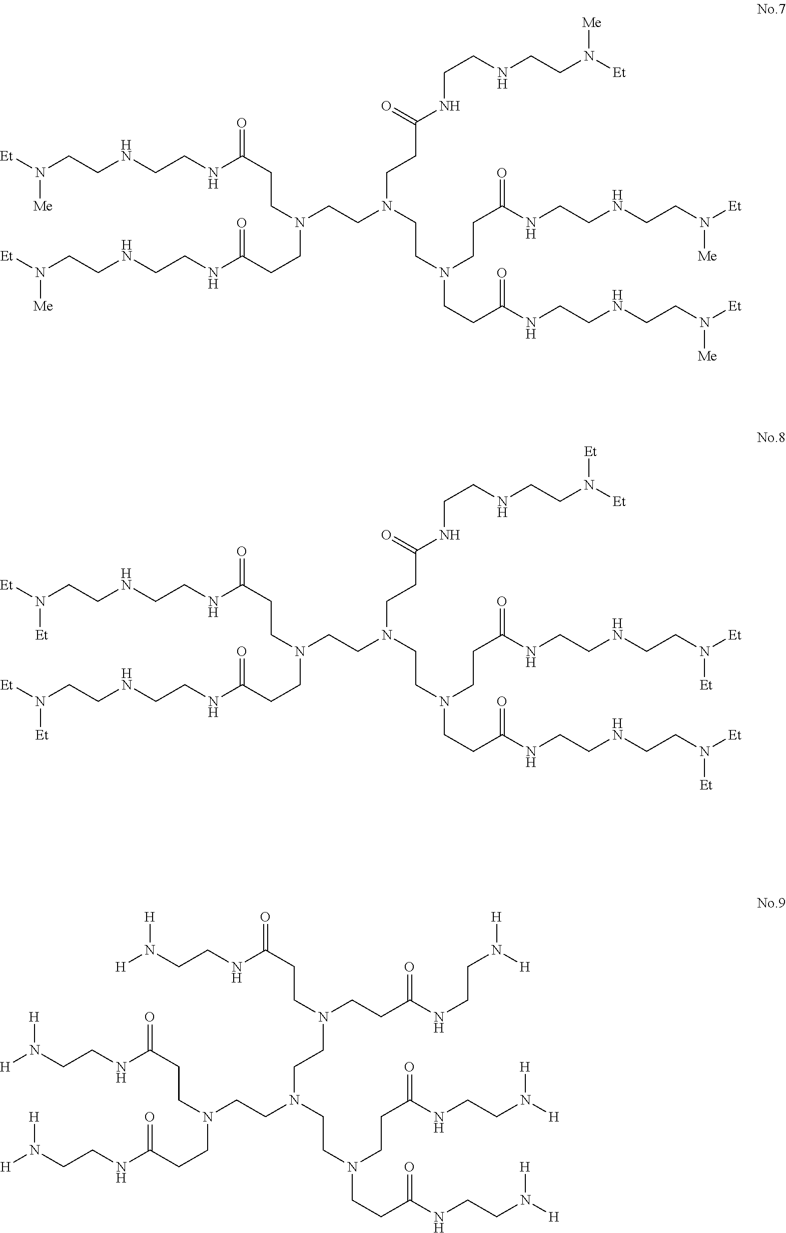

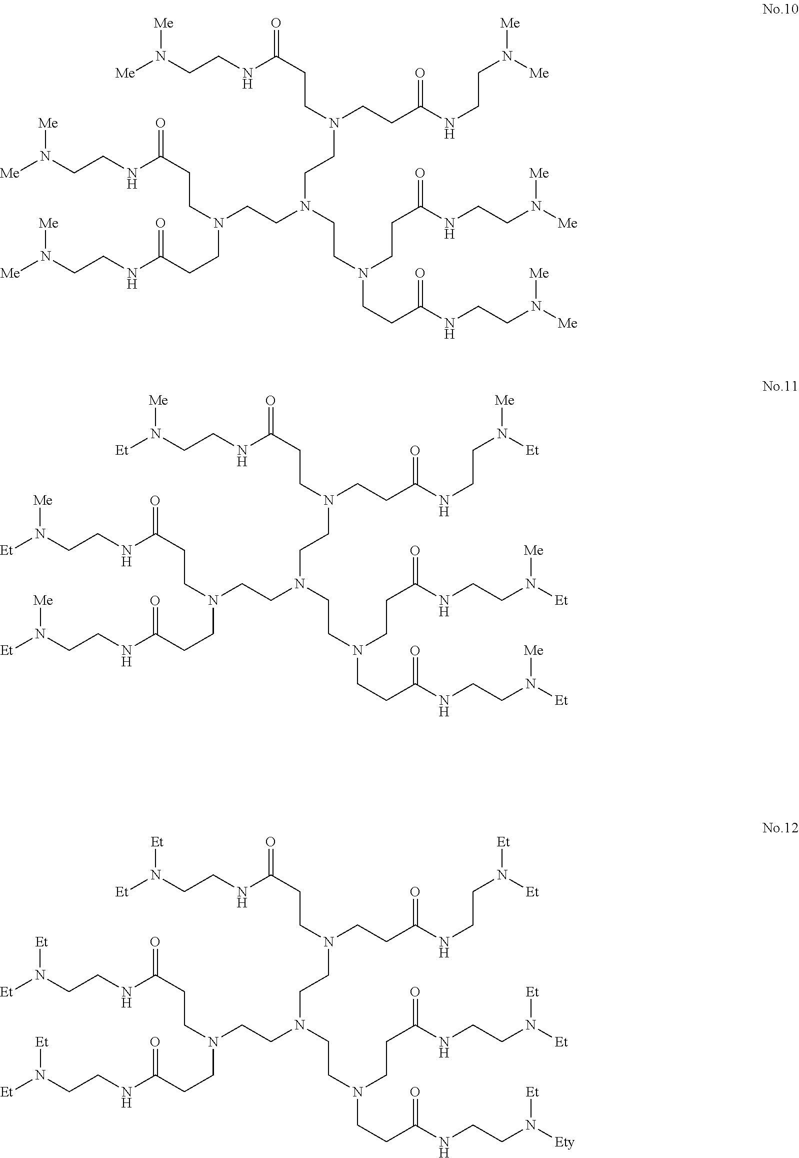

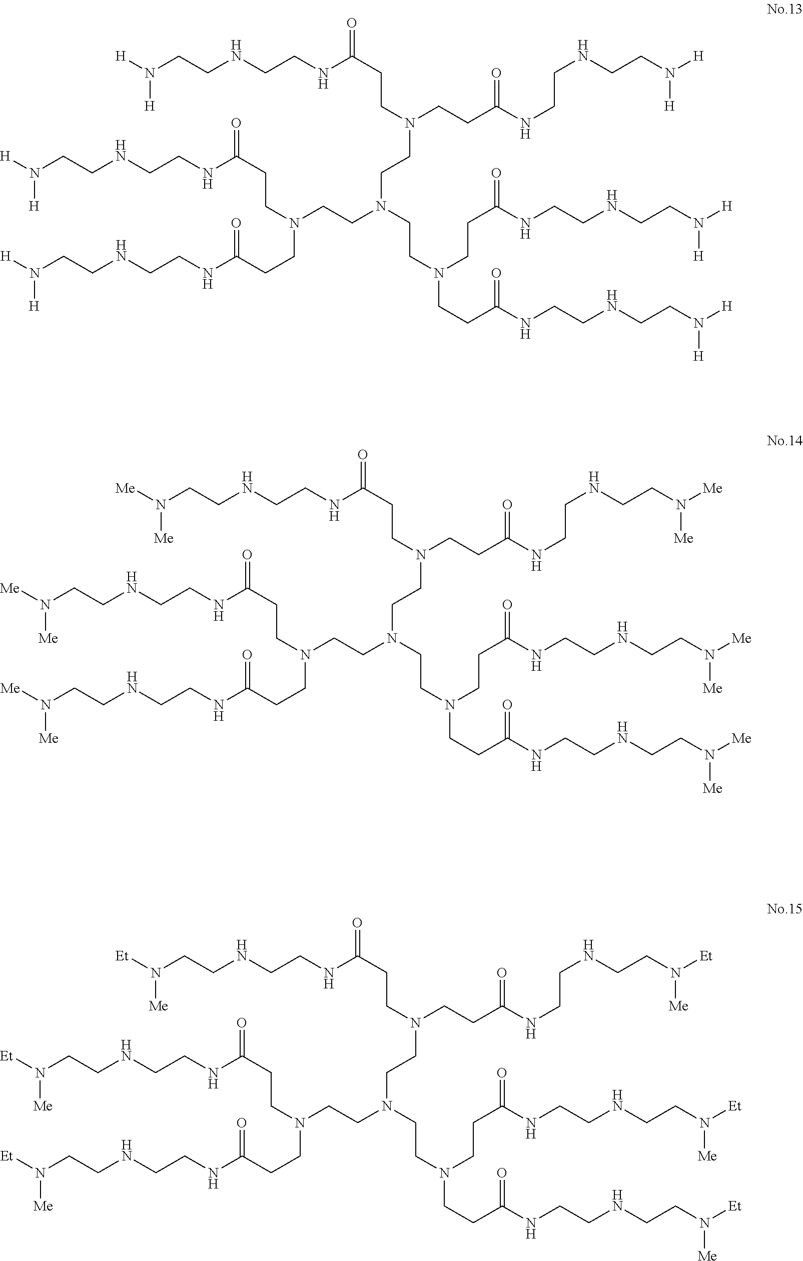

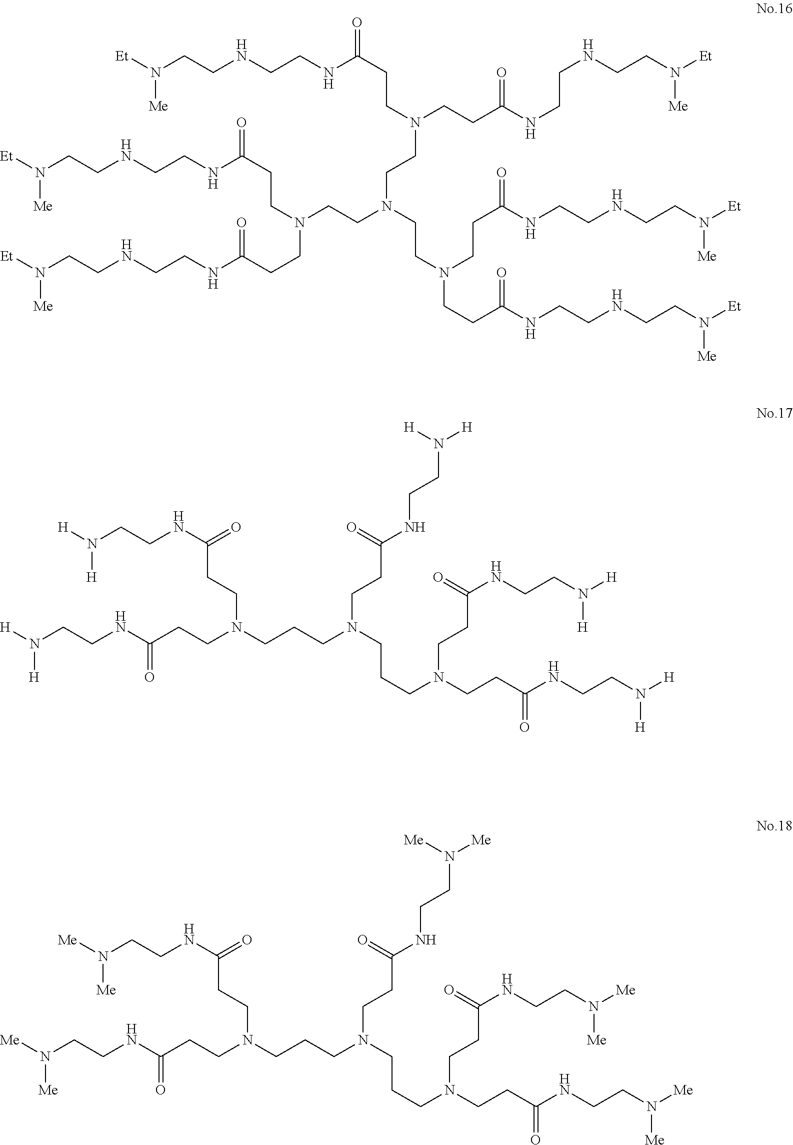

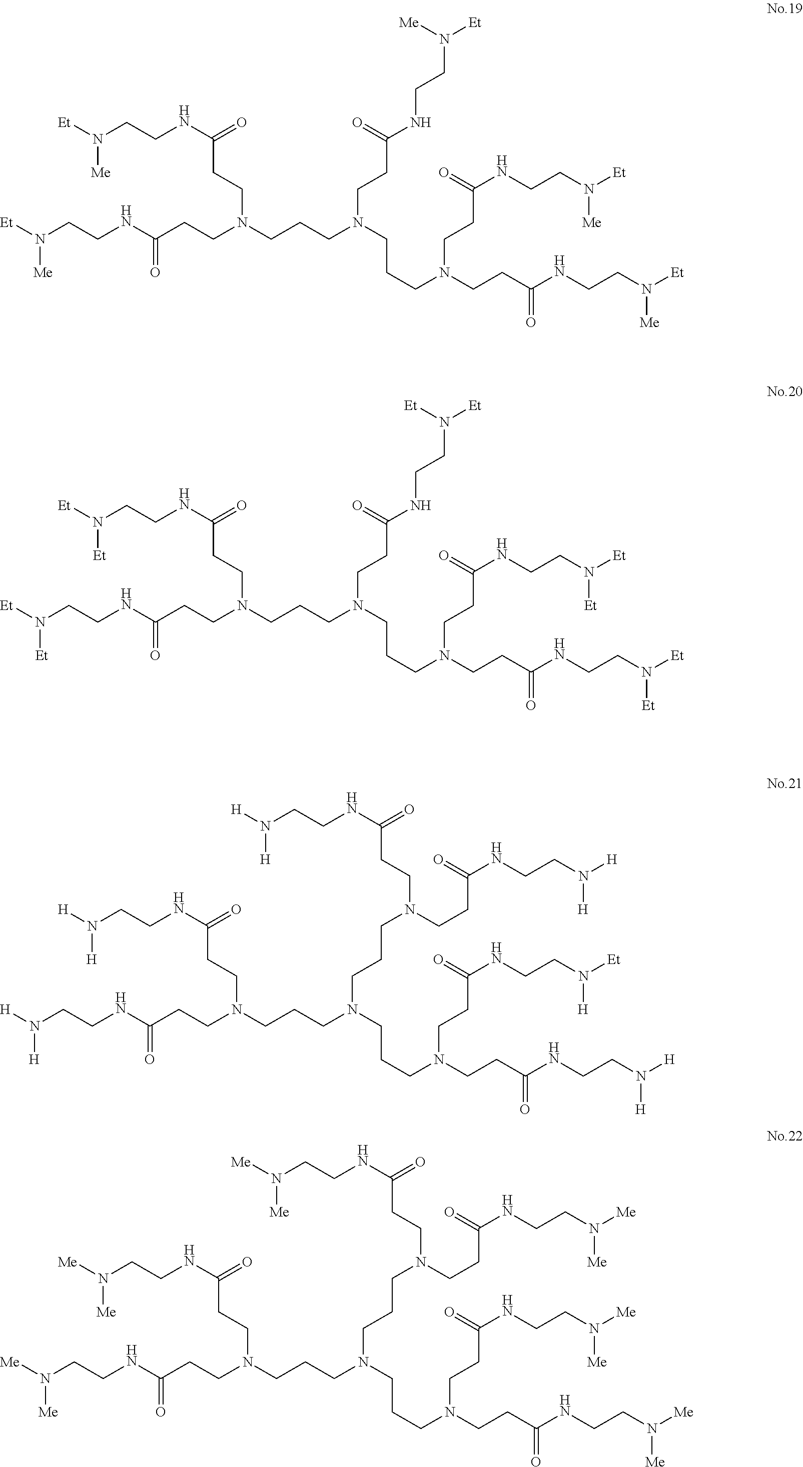

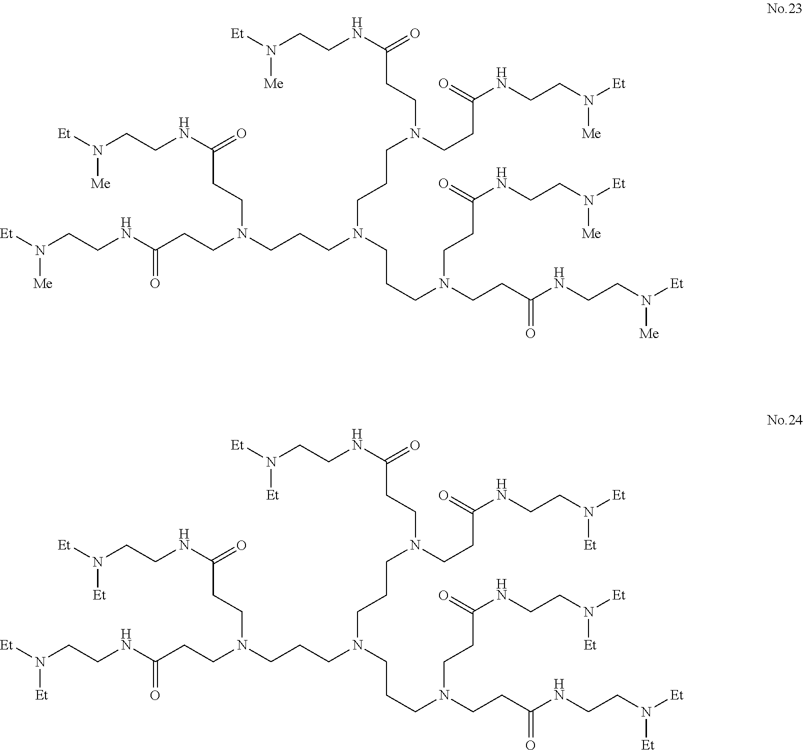

A method of producing the compound represented by the general formula (1) is not particularly limited, and the compound is produced by applying a well-known reaction. The compound represented by the general formula (1) can be obtained by, for example, causing a methyl alkenoate and an amine compound having a corresponding structure to react with each other, and then further causing an amine compound having another corresponding structure to react with the resultant. Specifically, Compound No. 1 can be obtained by, for example, causing methyl acrylate and diethylenetriamine to react with each other, and then further causing ethylenediamine to react with the resultant.

When a process for the formation of a metal layer on a base to be plated is performed by an electroplating method with an electroplating solution including the additive for an electroplating solution of the present invention, even in the case where the surface of the base to be plated has a fine structure, a metal can be filled in a groove or a hole in the surface with satisfactory surface flatness. Accordingly, a metal layer, which is suppressed from causing a defect having a depth of 10 μm or more in its side wall and is hence excellent in surface flatness, can be formed. In addition, when a process for the formation of a copper layer on the base to be plated is performed by the electroplating method with a copper electroplating solution including the additive for an electroplating solution of the present invention, the number of defects occurring in the side wall of a copper layer to be obtained is small, and hence a copper layer that is extremely excellent in surface flatness can be formed. Accordingly, the additive for an electroplating solution of the present invention is particularly suitable as an additive for a copper electroplating solution. In addition, even when a metal layer is formed at high speed by the electroplating method with the electroplating solution including the additive for an electroplating solution of the present invention, a metal layer, which is reduced in number of defects occurring in its side wall and is hence excellent in surface flatness, can be formed.

<Electroplating Solution>

Next, an electroplating solution of the present invention is described. The electroplating solution of the present invention is an aqueous solution including, as an essential effective component, an additive for an electroplating solution containing a compound represented by the general formula (1). From the viewpoint of further improving the effect of the present invention, the concentration of the compound represented by the general formula (1) in the electroplating solution is preferably from 0.01 mg/L to 100 mg/L, more preferably from 0.1 mg/L to 30 mg/L, most preferably from 0.5 mg/L to 10 mg/L.

In addition, to further improve the surface flatness of a metal layer formed by an electroplating method, the electroplating solution of the present invention preferably includes at least one kind of alcohol compound selected from methanol, ethanol, n-propanol, and isopropanol. Of the alcohol compounds, methanol is preferred because a metal layer that is particularly excellent in surface flatness can be formed. The alcohol compound is blended in an amount of preferably from 1 g to 100 g, more preferably from 5 g to 50 g with respect to 1 g of the compound represented by the general formula (1).

As in a conventionally known electroplating solution, the electroplating solution of the present invention may include, as components except the additive for an electroplating solution, a metal salt that is a metal supply source and an electrolyte, and a chloride ion source, a plating promoter, a plating inhibitor, or the like.

The metal of the metal salt to be used in the electroplating solution of the present invention is not particularly limited as long as the metal can be formed into a film by an electroplating method, and examples thereof include copper, tin, and silver. In particular, when the electroplating solution of the present invention is used as a copper electroplating solution, a copper layer excellent in surface flatness can be formed. Examples of the copper salt to be blended in the copper electroplating solution include copper sulfate, copper acetate, copper fluoroborate, and copper nitrate.

In addition, examples of an inorganic acid serving as the electrolyte to be used in the electroplating solution of the present invention include sulfuric acid, phosphoric acid, nitric acid, hydrogen halides, sulfamic acid, boric acid, and fluoroboric acid.

A case in which the electroplating solution of the present invention includes copper sulfate as the metal salt and sulfuric acid as the electrolyte is particularly preferred because a copper layer that is extremely excellent in surface flatness can be formed. In this case, from the viewpoint of suppressing the occurrence of a defect in the side wall of the copper layer, the concentration of copper sulfate (as CuSO₄.5H₂O) in the electroplating solution is preferably from 50 g/L to 500 g/L, more preferably from 100 g/L to 350 g/L, and the concentration of sulfuric acid in the electroplating solution is preferably from 20 g/L to 400 g/L, more preferably from 30 g/L to 150 g/L.

In addition, the electroplating solution of the present invention may be blended with a chloride ion source for forming a uniform and smooth metal layer. The concentration of the chloride ion source in the electroplating solution is preferably from 5 mg/L to 200 mg/L, more preferably from 20 mg/L to 150 mg/L. Although the chloride ion source is not particularly limited, examples thereof include hydrogen chloride and sodium chloride.

Further, the electroplating solution of the present invention may be blended with a plating promoter (brightening agent), such as an organic compound containing a sulfur element or a salt compound thereof. Examples of the plating promoter include compounds represented by the following general formulae (5) to (7).

XO₃S—R—SH (5)

XO₃—Ar—S—S—Ar—SO₃X (6)

In the general formulae (5) and (6), R represents an alkyl group that may be optionally substituted, preferably an alkyl group having 1 to 6 carbon atoms, more preferably an alkyl group having 1 to 4 carbon atoms, Ar represents an aryl group that may be optionally substituted, such as a phenyl group or a naphthyl group that may be optionally substituted, and X represents a counterion, such as a sodium or potassium ion.

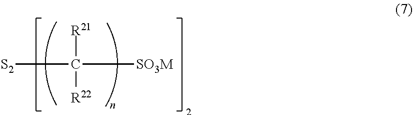

In the general formula (7), R²¹ and R²² each represent a hydrogen atom, a linear or branched alkyl group having 1 to 6 carbon atoms, a cycloalkyl group having 5 to 9 carbon atoms that may have a substituent having 1 to 3 carbon atoms, or an aryl group that may have a substituent having 1 to 3 carbon atoms, M represents an alkali metal, ammonium, or a monovalent organic ammonium, and “n” represents a number of from 1 to 7.

Of those described above, sodium 3,3′-dithiobis(1-propanesulfonate) (hereinafter sometimes abbreviated as “SPS”) is preferred as the plating promoter from the viewpoint that SPS has a high promoting effect on the formation of a metal layer.

The concentration of such plating promoter in the electroplating solution is preferably from 0.1 mg/L to 100 mg/L, more preferably from 0.3 mg/L to 50 mg/L, most preferably from 0.5 mg/L to 10 mg/L.

Further, the electroplating solution of the present invention is preferably blended with a plating inhibitor. For example, an oxygen-containing high-molecular weight organic compound may be used as the plating inhibitor. Specific examples thereof include polyethylene glycol, polypropylene glycol, a polyoxyethylene-polyoxypropylene random copolymer, and a polyoxyethylene-polyoxypropylene block copolymer. Of those, polyethylene glycol is preferred. From the viewpoint of further improving the effect of the present invention, the molecular weight of such oxygen-containing high-molecular weight organic compound is preferably from 500 to 100,000, more preferably from 1,000 to 10,000. In particular, polyethylene glycol having a molecular weight of from 1,000 to 10,000 is most preferred. From the same viewpoint, the concentration of the oxygen-containing high-molecular weight organic compound in the electroplating solution is preferably from 50 mg/L to 5,000 mg/L, more preferably from 100 mg/L to 3,000 mg/L.

Any other additive known to be capable of being added to a plating solution may be optionally used in the electroplating solution of the present invention to the extent that the effect of the present invention is not inhibited.

Examples of the other additive include an anthraquinone derivative, a cationic surfactant, a nonionic surfactant, an anionic surfactant, an amphoteric surfactant, an alkanesulfonic acid, an alkanesulfonic acid salt, an alkanesulfonic acid ester, a hydroxyalkanesulfonic acid, a hydroxyalkanesulfonic acid salt, a hydroxyalkanesulfonic acid ester, and a hydroxyalkanesulfonic acid organic acid ester. The concentration of such other additive in the electroplating solution is preferably from 0.1 mg/L to 500 mg/L, more preferably from 0.5 mg/L to 100 mg/L.

<Electroplating Method>

Next, an electroplating method including using the electroplating solution of the present invention is described. The electroplating method of the present invention only needs to be performed in the same manner as in a conventional electroplating method except that the electroplating solution of the present invention is used as an electroplating solution. Herein, a copper electroplating method including forming a copper layer on a base to be plated is described.

For example, a paddle stirring-type plating apparatus only needs to be used as an electroplating apparatus. The plating tank of the electroplating apparatus is filled with the copper electroplating solution of the present invention, and the base to be plated is immersed in the copper electroplating solution. For example, a product obtained by forming a resist pattern on a Si substrate with a copper seed layer through use of a photoresist may be used as the base to be plated.

At this time, for example, the temperature of the copper electroplating solution is from 10° C. to 70° C., preferably from 20° C. to 60° C., and a current density falls within the range of from 1 A/dm² to 70 A/dm², preferably from 5 A/dm² to 50 A/dm², more preferably from 15 A/dm² to 35 A/dm². In addition, for example, air stirring, quick liquid current stirring, or mechanical stirring with a stirring blade or the like may be used as a method of stirring the electroplating solution.

When copper is filled in an opening portion of the resist pattern under such conditions as described above, a copper layer, which is reduced in number of defects occurring in its side wall and is hence excellent in surface flatness, can be formed on the base to be plated.

A plated product to be manufactured by using the electroplating method of the present invention is not particularly limited, and examples thereof include a wide range of products such as materials for automobile industry (such as a heat sink, a carburetor part, a fuel injector, a cylinder, various valves, and an inner part of an engine), materials for electronic industry (such as contact, a circuit, a semiconductor package, a printed board, a film resistor, a capacitor, a hard disk, a magnetic material, a lead frame, a nut, a magnet, a resistor, a stem, a computer part, an electronic part, a laser oscillation device, an optical memory device, an optical fiber, a filter, a thermistor, a heater, a heater for high temperature, a varistor, a magnetic head, various sensors (gas, temperature, humidity, light, speed, and the like), and MEMS), precision instruments (such as a copying machine part, an optical instrument part, and a timepiece part), aviation or ship materials (such as an instrument of a hydraulic system, a screw, an engine, and a turbine), materials for chemical industry (such as a ball, a gate, a plug, and a check), various dies, a machine tool part, and a vacuum apparatus part. The electroplating method of the present invention is preferably used for the materials for electronic industry, in which a particularly fine pattern is required, is more preferably used in the manufacture of, among the materials, a semiconductor package and a printed board typified by TSV formation, bump formation, and the like, and is most preferably used in the semiconductor package.

A novel compound of the present invention is a compound represented by the general formula (3), and is suitable as an additive for an electroplating solution because when the compound is added to an electroplating solution, a metal layer, which is reduced in number of defects occurring in its side wall and hence has satisfactory surface flatness, is obtained. In addition, the novel compound of the present invention is particularly suitable as an additive for a copper electroplating solution because when the compound is added to a copper electroplating solution, the number of defects occurring in the side wall of a copper layer to be obtained is small, and hence the layer has particularly satisfactory surface flatness.

In the general formula (3), R¹¹ to R¹³ each independently represent a group represented by the general formula (4), A¹¹ represents an alkanediyl group having 2 to 4 carbon atoms, and “p” represents 0 or 1. Examples of the alkanediyl group having 2 to 4 carbon atoms that is represented by A¹¹ include an ethylene group, a propylene group, and a butylene group. All preferably represents an ethylene group or a propylene group because a metal layer that is more excellent in surface flatness can be formed.

In the general formula (4), R¹⁴ and R¹⁵ each independently represent a hydrogen atom or an alkyl group having 1 to 4 carbon atoms, A¹² and A¹³ each independently represent an alkanediyl group having 2 to 4 carbon atoms, “q” represents an integer of from 1 to 4, and * represents a bonding site, provided that when Au represents an alkanediyl group having 2 carbon atoms, “q” represents an integer of from 2 to 4. Examples of the alkyl group having 1 to 4 carbon atoms that is represented by any one of R¹⁴ and R¹⁵ include a methyl group, an ethyl group, a n-propyl group, an isopropyl group, a n-butyl group, an isobutyl group, and a tertiary butyl group. Examples of the alkanediyl group having 2 to 4 carbon atoms that is represented by any one of A¹² and A¹³ include an ethylene group, a propylene group, and a butylene group. R¹⁴ and R¹⁵ each preferably represent a hydrogen atom or a methyl group, and A¹² and A¹³ each preferably represent an ethylene group because a metal layer that is more excellent in surface flatness can be formed.

Examples of the novel compound represented by the general formula (3) of the present invention include the above-mentioned Compounds No. 5 to No. 8 and No. 13 to No. 24.

A method of producing the novel compound represented by the general formula (3) of the present invention is not particularly limited, and the compound is produced by applying a well-known reaction. The novel compound represented by the general formula (3) can be obtained by, for example, causing a methyl alkenoate and an amine compound having a corresponding structure to react with each other, and then further causing an amine compound having another corresponding structure to react with the resultant. Specifically, Compound No. 13 can be obtained by, for example, causing methyl acrylate and tris(2-aminoethyl)amine to react with each other, and then further causing diethylenetriamine to react with the resultant.

### EXAMPLES

Now, the present invention is described in more detail by way of Examples and Comparative Examples. However, the present invention is by no means limited by the following Examples and the like.

### Synthesis Example 1

### Synthesis of Compound No. 1

Under an Ar atmosphere, methanol (24.2 g) and methyl acrylate (12.8 g) were loaded into a 200-milliliter three-necked flask, and were sufficiently mixed. The solution was cooled to 0° C., and then a mixture of diethylenetriamine (2.6 g) and methanol (23.9 g) was dropped into the solution under the Ar atmosphere. The mixture was stirred at room temperature for 48 hours, and then methanol and an unreacted product were removed under reduced pressure in an oil bath at 60° C. Thus, an intermediate was obtained. Under the Ar atmosphere, methanol (48.2 g) and ethylenediamine (41.1 g) were loaded into a 300-milliliter three-necked flask, and were sufficiently mixed. The solution was cooled to 0° C., and then a mixture of the intermediate (5.0 g) and methanol (24.0 g) was dropped into the solution under the Ar atmosphere. The mixture was stirred at room temperature for 72 hours, and then methanol and an unreacted product were removed under reduced pressure in an oil bath at 60° C. Thus, a product was obtained. As a result of ¹H-NMR and elemental analysis, the resultant product was identified as Compound No. 1. Those analysis results are described below.

(Analysis Value)

(1) ¹H-NMR (D₂O)

3.25 ppm (10H, t), 2.82 ppm (10H, t), 2.72 ppm (10H, t), 2.63 ppm (8H, s), 2.45 ppm (10H, t)

### (2) Elemental Analysis (Theoretical Value)

C: 51.9 mass % (51.69 mass %), H: 9.2 mass % (9.42 mass %), N: 27.2 mass % (27.02 mass %), O: 11.7 mass % (11.87 mass %)

### Synthesis Example 2

### Synthesis of Compound No. 2

A product was obtained under the same conditions as those of Synthesis Example 1 except that N,N-dimethylethylenediamine was used instead of ethylenediamine. As a result of ¹H-NMR and elemental analysis, the resultant product was identified as Compound No. 2. Those analysis results are described below.

(Analysis Value)

(1) ¹H-NMR (D₂O)

3.32 ppm (10H, t), 2.80 ppm (10H, t), 2.61 ppm (8H, s), 2.51 ppm (10H, t), 2.42 ppm (10H, t), 2.25 ppm (30H, s)

### (2) Elemental Analysis (Theoretical Value)

C: 57.8 mass % (57.53 mass %), H: 10.0 mass % (10.28 mass %), N: 22.4 mass % (22.36 mass %), O: 9.8 mass % (9.83 mass %)

### Example 1

### Synthesis of Compound No. 5

A product was obtained under the same conditions as those of Synthesis Example 1 except that diethylenetriamine was used instead of ethylenediamine. As a result of ¹H-NMR and elemental analysis, the resultant product was identified as Compound No. 5. Those analysis results are described below.

(Analysis Value)

(1) ¹H-NMR (D₂O)

3.32 ppm (10H, t), 2.82 ppm (10H, t), 2.68 ppm (38H, m), 2.44 ppm (10H, t)

### (2) Elemental Analysis (Theoretical Value)

C: 52.8 mass % (52.68 mass %), H: 9.7 mass % (9.98 mass %), N: 28.3 mass % (28.35 mass %), O: 9.2 mass % (9.00 mass %)

### Synthesis Example 3

### Synthesis of Compound No. 9

A product was obtained under the same conditions as those of Synthesis Example 1 except that tris(2-aminoethyl)amine was used instead of diethylenetriamine. As a result of ¹H-NMR and elemental analysis, the resultant product was identified as Compound No. 9. Those analysis results are described below.

(Analysis Value)

(1) ¹H-NMR (D₂O)

3.25 ppm (12H, t), 2.82 ppm (12H, t), 2.72 ppm (12H, t), 2.64 ppm (12H, s), 2.45 ppm (12H, t)

### (2) Elemental Analysis (Theoretical Value)

C: 52.3 mass % (52.03 massa), H: 9.2 mass % (9.46 mass %), N: 27.0 mass % (26.96 mass %), O: 11.5 mass % (11.55 mass %)

### Synthesis Example 4

### Synthesis of Compound No. 10

A product was obtained under the same conditions as those of Synthesis Example 3 except that N,N-dimethylethylenediamine was used instead of ethylenediamine. As a result of ¹H-NMR and elemental analysis, the resultant product was identified as Compound No. 10. Those analysis results are described below.

(Analysis Value)

(1) ¹H-NMR (D₂O)

3.32 ppm (12H, t), 2.80 ppm (12H, t), 2.62 ppm (12H, s), 2.50 ppm (12H, t), 2.42 ppm (12H, t), 2.24 ppm (36H, s)

### (2) Elemental Analysis (Theoretical Value)

C: 57.9 mass % (57.68 mass %), H: 10.0 mass % (10.29 mass %), N: 22.4 mass % (22.42 mass %), O: 9.7 mass % (9.60 mass %)

### Example 2

### Synthesis of Compound No. 13

A product was obtained under the same conditions as those of Synthesis Example 3 except that diethylenetriamine was used instead of ethylenediamine. As a result of ¹H-NMR and elemental analysis, the resultant product was identified as Compound No. 13. Those analysis results are described below.

(Analysis Value)

(1) ¹H-NMR (D₂O)

3.32 ppm (12H, t), 2.82 ppm (12H, t), 2.67 ppm (48H, m), 2.44 ppm (12H, t)

### (2) Elemental Analysis (Theoretical Value)

C: 53.2 mass % (52.92 mass %), H: 9.6 mass % (9.99 mass %), N: 28.1 mass % (28.28 mass %), O: 9.1 mass % (8.81 mass %)

### Example 3

### Synthesis of Compound No. 17

A product was obtained under the same conditions as those of Synthesis Example 1 except that dipropylenetriamine was used instead of diethylenetriamine. As a result of ¹H-NMR and elemental analysis, the resultant product was identified as Compound No. 17. Those analysis results are described below.

(Analysis Value)

(1) ¹H-NMR (D₂O)

3.24 ppm (10H, t), 2.80 ppm (10H, t), 2.72 ppm (10H, t), 2.46 ppm (18H, m), 1.65 ppm (4H, m)

### (2) Elemental Analysis (Theoretical Value)

C: 53.2 mass % (53.04 mass %), H: 9.5 mass % (9.62 mass %), N: 26.0 mass % (25.94 mass %), O: 11.3 mass % (11.40 mass %)

### Example 4

### Synthesis of Compound No. 21

A product was obtained under the same conditions as those of Production Example 1 except that tris(3-aminopropyl)amine was used instead of diethylenetriamine. As a result of ¹H-NMR and elemental analysis, the resultant product was identified as Compound No. 21. Those analysis results are described below.

(Analysis Value)

(1) ¹H-NMR (D₂O)

3.24 ppm (12H, t), 2.80 ppm (12H, t), 2.72 ppm (12H, t), 2.46 ppm (24H, m), 1.63 ppm (6H, m)

### (2) Elemental Analysis (Theoretical Value)

C: 53.8 mass % (53.64 mass %), H: 9.5 mass % (9.70 mass %), N: 25.6 mass % (25.67 mass %), O: 11.1 mass % (10.99 mass %)

### Examples 5 to 15

Copper electroplating solutions were prepared according to compositions shown in Table 1 by using Compounds No. 1, No. 2, No. 5, No. 9, No. 10, No. 13, No. 17, and No. 21 as additives for electroplating solutions. In each of Examples 5 to 15, the solvent of the copper electroplating solution was water, and the concentration of each component was adjusted with water. In addition, PEG4000 used in Examples is polyethylene glycol having a weight-average molecular weight of from 3,600 to 4,400.

TABLE 1

| | | Methanol | | | | | |
|---|---|---|---|---|---|---|---|
| Examples | | (amount with | | | | | |
| Copper | | respect to 1 g | Copper sulfate | Sulfuric | Hydrogen | | |
| plating | Added compound | of added | pentahydrate | acid | chloride | SPS | PEG4000 |
| solution | (concentration) | compound) | (g/L) | (g/L) | (mg/L) | (mg/L) | (g/L) |
| Example 5 | Compound No. 1 | 20 g | 250 | 100 | 50 | 1 | 1 |
| | (1 mg/L) | | | | | | |
| Example 6 | Compound No. 1 | 0 g | 250 | 100 | 50 | 1 | 1 |
| | (10 mg/L) | | | | | | |
| Example 7 | Compound No. 2 | 20 g | 250 | 100 | 50 | 1 | 1 |
| | (1 mg/L) | | | | | | |
| Example 8 | Compound No. 2 | 0 g | 250 | 100 | 50 | 1 | 1 |
| | (1 mg/L) | | | | | | |
| Example 9 | Compound No. 5 | 0 g | 250 | 100 | 50 | 1 | 1 |
| | (1 mg/L) | | | | | | |
| Example 10 | Compound No. 9 | 4 g | 250 | 100 | 50 | 1 | 1 |
| | (5 mg/L) | | | | | | |
| Example 11 | Compound No. 9 | 0 g | 250 | 100 | 50 | 1 | 1 |
| | (1 mg/L) | | | | | | |
| Example 12 | Compound No. 10 | 0 g | 250 | 100 | 50 | 1 | 1 |
| | (1 mg/L) | | | | | | |
| Example 13 | Compound No. 13 | 0 g | 250 | 100 | 50 | 1 | 1 |
| | (1 mg/L) | | | | | | |
| Example 14 | Compound No. 17 | 0 g | 250 | 100 | 50 | 1 | 1 |
| | (1 mq/L) | | | | | | |
| Example 15 | Compound No. 21 | 0 g | 250 | 100 | 50 | 1 | 1 |
| | (1 mg/L) | | | | | | |

### Comparative Examples 1 to 3

Copper electroplating solutions were prepared according to compositions shown in Table 2 by using the following comparative compounds 1 and 2 as additives for electroplating solutions. In each of Comparative Examples 1 to 3, the solvent of the copper electroplating solution was water, and the concentration of each component was adjusted with water. In addition, PEG4000 used in Comparative Examples is polyethylene glycol having a weight-average molecular weight of from 3,600 to 4,400.

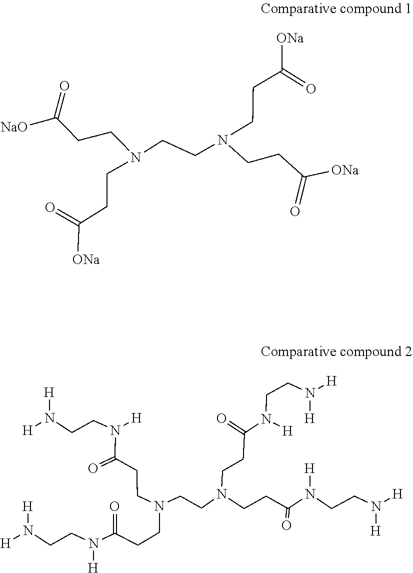

TABLE 2

| Comparative | | Methanol | | | | | |
|---|---|---|---|---|---|---|---|
| Examples | | (amount with | | | | | |
| Copper | Added | respect to 1 g | Copper sulfate | Sulfuric | Hydrogen | | |
| plating | compound | of added | pentahydrate | acid | chloride | SPS | PEG4000 |
| solution | (concentration) | compound) | (g/L) | (g/L) | (mg/L) | (mg/L) | (g/L) |
| Comparative | Comparative | 0 g | 250 | 100 | 50 | 1 | 1 |
| Example 1 | compound 1 | | | | | | |
| | (1 mg/L) | | | | | | |
| Comparative | Comparative | 20 g | 250 | 100 | 50 | 1 | 1 |
| Example 2 | compound 2 | | | | | | |
| | (1 mg/L) | | | | | | |
| Comparative | Comparative | 0 g | 250 | 100 | 50 | 1 | 1 |
| Example 3 | compound 2 | | | | | | |
| | (1 mg/L) | | | | | | |

### Evaluation Examples 1 to 22 and Comparative Evaluation Examples 1 to 6

A paddle stirring-type plating apparatus was used as an electroplating apparatus, and the plating tank of the paddle stirring-type plating apparatus was filled with each of the copper electroplating solutions of Examples 5 to 15 and Comparative Examples 1 to 3. A base to be plated was immersed in each of the copper electroplating solutions. A product obtained by forming a resist pattern (shape: having an opening portion of a circular sectional shape, opening diameter: 200 μm) on a Si substrate with a copper seed layer through use of a photoresist was used as the base to be plated. Next, copper of each copper electroplating solution was filled in the opening portion of the resist under the following plating conditions by a copper electroplating method. Thus, a copper layer was formed on the base to be plated.

### (Plating Conditions)

(1) Hole diameter: 200 μm

(2) Current density: 20 A/dm² or 25 A/dm²

(3) Liquid temperature: 45° C.

(4) Plating time: A time period required for the minimum height (L_{Min}) of a copper layer to become 200 μm

### Evaluation Examples 1 to 22 and Comparative Evaluation Examples 1 to 6

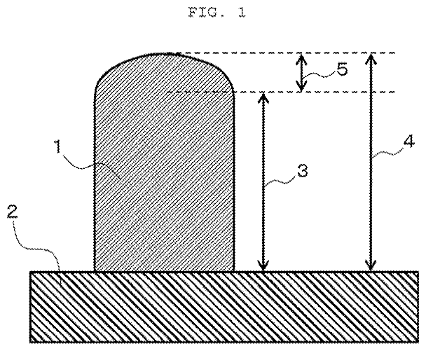

The minimum height (L_{Min}) and maximum height (L_{Max}) of a copper layer 1 formed on the surface of a base 2 to be plated by each of Evaluation Examples 1 to 22 and Comparative Evaluation Examples 1 to 6 as illustrated in FIG. 1 were measured by observing a section of the copper layer 1 with a laser microscope (manufactured by Keyence Corporation, model number: VK-9700), and ΔL was calculated from the following equation. In addition, when a recess having a depth of 10 μm or more was observed in the side wall of the copper layer 1, the recess was defined as a defect, and the depth of the defect was measured. Evaluation results are shown in Table 3.

ΔL=L_{Max}−L_{Min}

TABLE 3

| | | | | Presence | Depth |
|---|---|---|---|---|---|
| | Copper | Current | | or | of |
| | electroplating | density | ΔL | absence | defect |
| | solution | (A/dm²) | (μm) | of defect | (μm) |
| Evaluation Example 1 | Example 5 | 25 | 10 | Absent | — |
| Evaluation Example 2 | | 20 | 10 | Absent | — |
| Evaluation Example 3 | Example 6 | 25 | 15 | Absent | — |
| Evaluation Example 4 | | 20 | 10 | Absent | — |
| Evaluation Example 5 | Example 7 | 25 | 15 | Absent | — |
| Evaluation Example 6 | | 20 | 15 | Absent | — |
| Evaluation Example 7 | Example 8 | 25 | 20 | Absent | — |
| Evaluation Example 8 | | 20 | 15 | Absent | — |
| Evaluation Example 9 | Example 9 | 25 | 15 | Absent | — |
| Evaluation Example 10 | | 20 | 15 | Absent | — |
| Evaluation Example 11 | Example 10 | 25 | 15 | Absent | — |
| Evaluation Example 12 | | 20 | 10 | Absent | — |
| Evaluation Example 13 | Example 11 | 25 | 10 | Absent | — |
| Evaluation Example 14 | | 20 | 10 | Absent | — |
| Evaluation Example 15 | Example 12 | 25 | 15 | Absent | — |
| Evaluation Example 16 | | 20 | 10 | Absent | — |
| Evaluation Example 17 | Example 13 | 25 | 20 | Absent | — |
| Evaluation Example 18 | | 20 | 15 | Absent | — |
| Evaluation Example 19 | Example 14 | 25 | 20 | Absent | — |
| Evaluation Example 20 | | 20 | 15 | Absent | — |
| Evaluation Example 21 | Example 15 | 25 | 15 | Absent | — |
| Evaluation Example 22 | | 20 | 10 | Absent | — |
| Comparative Evaluation | Com- | 25 | 40 | Present | 40 |
| Example 1 | parative | | | | |
| Comparative Evaluation | Example 1 | 20 | 35 | Present | 20 |
| Example 2 | | | | | |
| Comparative Evaluation | Com- | 25 | 45 | Present | 25 |
| Example 3 | parative | | | | |
| Comparative Evaluation | Example 2 | 20 | 35 | Present | 10 |
| Example 4 | | | | | |
| Comparative Evaluation | Com- | 25 | 50 | Present | 30 |
| Example 5 | parative | | | | |
| Comparative Evaluation | Example 3 | 20 | 35 | Present | 10 |
| Example 6 | | | | | |

In Table 3, a smaller value of ΔL means that a copper layer that was more excellent in surface flatness was able to be formed. It was found from the results of Table 3 that in each of Evaluation Examples 1 to 22, a copper layer, which was excellent in surface flatness and was free of any defect in its side wall as compared to those of Comparative Evaluation Examples 1 to 6, was able to be formed. Further, it was found that in the case where any one of the copper electroplating solutions of Comparative Examples 1 to 3 was used, when the current density was increased from 20 A/dm² to 25 A/dm², the surface flatness of the copper layer deteriorated to increase the depth of a defect thereof. In contrast, it was found that in the case where any one of the copper electroplating solutions of Examples 5 to 15 was used, even when the current density was increased from 20 A/dm² to 25 A/dm², the copper layer maintained satisfactory surface flatness to prevent the occurrence of a defect therein. The foregoing means that in the case where any one of the copper electroplating solutions of Examples 5 to 15 is used, a copper layer, which is reduced in number of defects occurring in its side wall and is hence excellent in surface flatness, can be obtained with satisfactory productivity as compared to the case where any one of the copper electroplating solutions of Comparative Examples 1 to 3 is used.

As described above, it was found that when a copper layer was formed on the base to be plated by the electroplating method with the electroplating solution including the additive for an electroplating solution of the present invention, a copper layer, which was reduced in number of defects occurring in its side wall and was hence excellent in surface flatness, was able to be formed.

### EXPLANATION ON NUMERALS

1 copper layer
2 base to be plated
3 minimum height (L_{Min})
4 maximum height (L_{Max})
5 ΔL

1. An additive for an electroplating solution, comprising a compound represented by the following general formula (1): 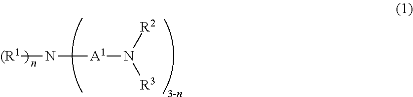
where R¹ to R³ each independently represent a group represented by the following general formula (2), A¹ represents an alkanediyl group having 2 to 4 carbon atoms, and “n” represents 0 or 1: 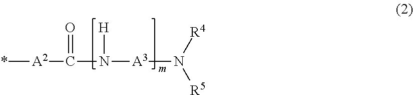
where R⁴ and R⁵ each independently represent a hydrogen atom or an alkyl group having 1 to 4 carbon atoms, A² and A³ each independently represent an alkanediyl group having 2 to 4 carbon atoms, “m” represents an integer of from 1 to 4, and * represents a bonding site.

2. The additive for an electroplating solution according to claim 1, wherein the additive for an electroplating solution is an additive for a copper electroplating solution.

3. An electroplating solution, comprising the additive for an electroplating solution of claim 1.

4. The electroplating solution according to claim 3, further comprising at least one kind of alcohol compound selected from methanol, ethanol, n-propanol, and isopropanol.

5. The electroplating solution according to claim 4, wherein the electroplating solution comprises 1 g to 100 g of the alcohol compound with respect to 1 g of the compound represented by the general formula (1).

6. The electroplating solution according to claim 3, further comprising:
a metal salt; and
an electrolyte.

7. The electroplating solution according to claim 6, wherein the metal salt is copper sulfate and the electrolyte is sulfuric acid.

8. The electroplating solution according to claim 3, further comprising a chloride ion source.

9. The electroplating solution according to claim 8, wherein the chloride ion source is hydrogen chloride.

10. An electroplating method, comprising using the electroplating solution of claim 3.

11. A compound represented by the following general formula (3): 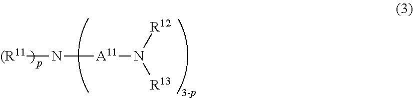
where R¹¹ to R¹³ each independently represent a group represented by the following general formula (4), A¹¹ represents an alkanediyl group having 2 to 4 carbon atoms, and “p” represents 0 or 1: 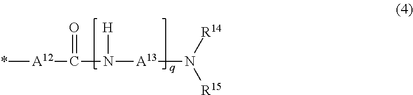
where R¹⁴ and R¹⁵ each independently represent a hydrogen atom or an alkyl group having 1 to 4 carbon atoms, A¹² and A¹³ each independently represent an alkanediyl group having 2 to 4 carbon atoms, “q” represents an integer of from 1 to 4, and * represents a bonding site, provided that when A¹¹ represents an alkanediyl group having 2 carbon atoms, “q” represents an integer of from 2 to 4.
